(19) United States
(12) Patent Application Publication
(10) Pub. No.: US 2004/0242863 A1
(43) Pub. Date: Dec. 2, 2004

(54) Preparation of cefalosporins intermediates and conversion of said intermediates into active compound precursors

(76) Inventor: Alessandro Riccardo Carenini, Milano (IT)

Correspondence Address: NIXON & VANDERHYE, PC, 1100 N GLEBE ROAD, 8TH FLOOR, ARLINGTON, VA, 22201-4714, US

(21) Appl. No.: 10/445,296

(22) Filed: May 27, 2003

(51) Int. Cl. C07D 501/14

(52) U.S. Cl. 540/222

(57) ABSTRACT

There is described the preparation of the new 7β-[(2-aminothiazol-4-yl)glyoxylamido-3-hydroxymethyl-2-cephem-4-carboxylic acid derivatives, in which the amino group is free or protected by a removable group, and the subsequent conversion of the compounds thus obtained into active cephalosporin precursors either by esterification of the hydroxy group or replacement of the hydroxy group by a halogen atom and, in the latter case, by subsequent replacement of the halogen atom of said 7β-[(2-aminothiazol-4-yl)glyoxylamido-3-halomethyl-3-cephem-4-carboxylic acid derivatives by the residue of a nucleophilic compound to obtain corresponding, immediate precursors of 7β-[α-(2-aminothiazol-4-yl-α-methoxyimino]acetamido-3-(substituted)methyl-3-cephem-4-carboxylic acid derivatives.

### OBJECT OF THE INVENTION

The present invention concerns novel 7δ-[(2-aminothiazol-4-yl)glyoxylamido-3-hydroxymethyl-3-cephem-4-carboxylic acid derivatives, a process for their preparation and for their conversion into active antibiotics. More particularly, the present invention concerns the preparation of the new 7β-[(2-aminothiazol-4-yl)glyoxylamido-3-hydroxymethyl-3-cephem-4-carboxylic acid, in which the amino group is free or protected by a removable group, and the subsequent conversion of the compound thus obtained into active cephalosporin precursors either by esterification of the hydroxy group or replacement of the hydroxy group by a leaving group and, in the latter case, by subsequent replacement of the leaving group by the residue of a nucleophilic compound to obtain corresponding, immediate precursors of 7β-[α-(2-aminothiazol-4-yl-α-methoxyimino]acetamido-3-(substituted)methyl-3-cephem-4-carboxylic acid derivatives.

### BACKGROUND OF THE INVENTION

Many semisynthetic, therapeutically used cephalosporins generally consist of 7β-acylamido-3-(substituted)methyl-3-cephem-4-carboxylic acid derivatives in which the 3-methyl group is substituted with the residue of a nucleophilic compound and the acyl group is a variously 2-mono-, 2,2-di- or 2,2,2-tri-substituted acetic acid. Mainly, the residue of the nucleophilic compound is a methoxy; acetoxy; carbamoyl; an optionally substituted heterocycle radical, including tertiary ammonium heterocycles forming an inner salt with the cephem-4-carboxylic acid, such as 5-methyltetrazol-2-yl, 1-pyridinio, 4-carbamoyl-1-pyridinio, 2-sulfoethyl-1-pyridinio, 5,6,7,8-tetrahydro-1-quinolinio, 1-cyclopentano[b]pyridinio, 1H-imidazo[1,2-b]-4-pyridazinio, 5-methyl-1-(2-hydroxyethyl)-3-imidazolio, 1-quinuclidinio, 4-carbamoly-1-quinuclidinio, 1-methyl-1-pyrrolidinio; or a mercapto group substituted with a furoyl, thenoyl group or with an optionally substituted heterocycle radical such as 1-methyltetrazol-5-yl, 1-sulfomethyltetrazol-5-yl, 1-(2-dimethylaminoethyl)tetrazol-5-yl, thiazol-2-yl, 5-carboxymethyl-4-methylthiazol-2-yl, 5-methyl-1,3,4thiadiazol-2-yl, 1H-1,2,3-triazol-4-yl, 1H-1,2,4-triazol-3-yl, 5-methyl-1,3,4-thiadiazol-2-yl, 1,2,5,6-tetrahydro-2-methyl-5,6-dioxo-1,2,4-triazin-3-yl.

Mainly, the so-called “third-generation-cephalosporins” generally consist of 3-methyl-3-cephem-4-carboxylic acid derivatives substituted in the 7β-position by an α-(2-aminothiazol-4-yl)-α-hydroxyiminoacetamido group, in which the hydroxy group is free or O-substituted with an alkyl group, optionally substituted by a carboxy group, and substituted on the methyl group in the 3-position with the residue of a nucleophilic compound.

The above mentioned third-generation-cephalosporins have been described for the first time in the published German application DE 2556736 (corresponding to U.S. Pat. No. 5,583,216) and, principally, are compounds encompassed by the general formula A

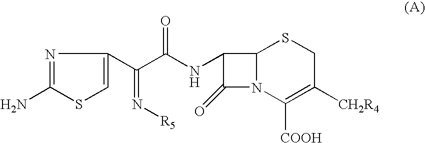

wherein R₄ is the residue of a nucleophilic compound and R₅ is a hydroxy group which may be protected, including their pharmaceutically acceptable esters, the respective salts and the relative solvates.

Among these third-generation-cephalosporins, the 7β-[(2-aminothiazol-4-yl)glyoxylamido-3-hydroxymethyl-3-cephem-4-carboxylic acid derivatives of formula A, in which R₅ is methoxy and R₄ is an acetoxy group (cefotaxime) or a 1,2,5,6-tetrahydro-2-methyl-5,6-dioxo-1,2,4-triazin-3-ylthio group (ceftriaxone) are particularly interesting compounds.

### DESCRIPTION OF THE PRIOR ART

The 7β-[α-(2-aminothiazol-4-yl)-α-hydroxyimino]acetamido-3-methyl-3-cephem-4-carboxylic acid derivatives in which the 3-methyl group is substituted with the residue of a nucleophilic compound are prepared according to the general method, described in DE 2556736 and U.S. Pat. No.5,583,216, which comprises introducing the α-(2-aminothiazol-4-yl)-α-hydroxyiminoacetyl radical, wherein the hydroxy and amino groups are suitably protected, onto the amino group of a 7β-amino-3-methylcephem-4-carboxylic acid in which the methyl group is substituted with the residue of a nucleophilic compound.

The 3-CH₂R₄ substituent in the formula A above is easily derivable from cephalosporins produced by fermentation and the meaning of R₄ is illustrated for example in U.S. Pat. No. 5,583,216.

Since the appearance of the document DE 2,556,736, in 1976, the sole valid process suitable for the preparation of the cephalosporins of formula A had been that described in said document. Recently, processes disclosing the introduction of a precursor of the α-(2-aminothiazol-4-yl)-α-hydroxyiminoacetyl radical onto the amino group of a 7β-amino-3-methyl-3-cephem in which the methyl group is substituted with the residue of a nucleophilic compound have been disclosed (see EP 842937 A2 and U.S. 2002/0128469 A1).

U.S. Pat. No. 4,201,779 discloses, among other compounds, cephem derivatives of formula B

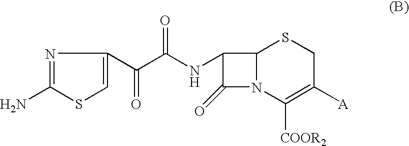

wherein R₂ denotes hydrogen, optionally substituted alkyl phthalide or cation and denotes hydrogen, alkoxy, halogen or a group —CH₂Y, in which Y represents hydrogen, acyloxy, alkoxy, optionally substituted carbamoyloxy or a group —SR₃, in which R₃ can represent acyl, alky, or an optionally substituted 5-membered or 6-membered heterocyclic structure, said cephem derivatives being valuable antibiotics. According to this document, the compounds of formula B are prepared by reaction of the N-protected 2-aminothiazol-4-ylglyoxylic acid with a corresponding 7β-amino-3-(A)substituted-3-cephem-4-carboxylate.

To make the terminology uniform and the text more understandable, in the present description meanings and conventional expressions, in singular or plural form, will be used. In particular, unless otherwise specified:

the term “7β-cephem” designates the radical of formula (a)

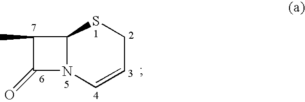

the oxyimino group which is present in the formula A above and in the other formulas hereinbelow is in the syn configuration;

“Alk” and designates an alkyl group of from 1 to 4 carbon atoms, the methyl and ethyl group being also referred to as “Me” and “Et”, respectively;

“alkane” indicates hydrocarbons of from 1 to 4 carbon atoms;

“arene” and “aryl” refer to benzene, toluene and their radicals.

### SUMMARY OF THE INVENTION

The present invention is based on the hypothesis that, starting from 7β-amino-3-hydroxymethyl-3-cephem-4-carboxylic acid, which is commercially available and directly obtainable in the cephalosporin C manufacture by introducing an esterase in the fermentation medium, it would be possible to prepare third-generation-cephalosporins by an easily conducible, few-step process.

Thus, it has been found that, by reacting 2-aminothiazol-4-ylglyoxylic acid with a silylated 7β-amino-3-hydroxymethyl-3-cephem-4-carboxylic acid, a new 7β-(2-aminothiazol-4-yl)glyoxylamido-3-hydroxymethyl-3-cephem-4-carboxylic acid is obtained in very good yields

It has also been found that this compound is a useful intermediate which, by optional previous protection of the free amino group and/or of the carboxyl group, may be converted either into a 7β-[(2-aminothiazol-4-yl)glyoxylamido]cephalosporanic acid derivative by reaction with acetic anhydride or into a 7β-[(2-aminothiazol-4-yl)glyoxylamido]-3-halomethyl-3-cephem-4-carboxylic acid derivative by introduction of a leaving group on the methyl in 3-position and reaction with the sodium or potassium salt of 1,2,5,6-tetrahydro-2-methyl-5,6-dioxo-1,2,4-triazin-3-thiol (2-methyl-5,6-dioxo-perihydro-1,2,4-triazine-3-thione) to obtain a corresponding 7β-[(2-aminothiazol-4-yl)glyoxylamido]-3-(1,2,5,6-tetrahydro-2-methyl-5,6-dioxo-1,2,4-triazin-3-thio)methyl-3-cephem-4-carboxylic acid. The 7β-[(2-aminothiazol-4-yl)gloxylamido]cephalosporanic acid and 7β-[(2-aminothiazol-4-yl)glyoxylamido]-3-(1,2,5,6,-tetrahydro-2-methyl-5,6-dioxo-1,2,4-triazin-3-thio)methyl-3-cephem-4-carboxylic acid derivatives are precursors of cefotaxime (formula A in which R₄=acetoxy and R₅=methoxy) and ceftriaxone (formula A in which R₄=1,2,5,6-tetrahydro-2-methyl-5,6-dioxo-1,2,4-triazin-3-thio and R₅=methoxy), which are obtained by reaction of said derivatives with methoxyamine and further deprotection, if needed.

### DETAILED DESCRIPTION OF THE INVENTION

Thus, it is an object of the present invention to provide novel 7β-(2-aminothiazol-4-yl)glyoxylamido-3-hydroxymethyl-3-cephem-4-carboxylic acid derivatives of formula I

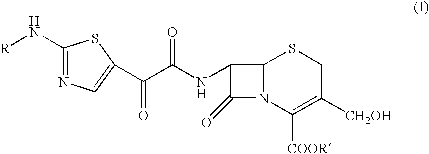

wherein R is hydrogen or a N-protective group Pr and R′ is hydrogen or a now-silylated carboxy protective group Pr′, and the salts of said derivatives of formula I in which at least one of R and R′ is hydrogen.

In particular, the invention provides the compounds of formula I above, wherein R and R′ are both hydrogen, and their salts with alkaline metals or with organic bases and their acid addition salts, or wherein R is hydrogen and R′ is a Pr′ group and their acid addition salts, or wherein R is a Pr group and R′ is hydrogen and their salts with alkaline metals or organic bases, or wherein R is a Pr group and R′ is a Pr′ group. All these compounds of formula I are useful intermediates in the synthesis of third-generation-cephalosporins, in particular of cefotaxime and cefotriaxone.

The N-protective group Pr may be selected among the groups commonly used in the peptide and beta-lactam chemistry such as, for example, formyl, t-butoxycarbonyl, benxylozycarbonyl, diphenylmethyl, triphenylmethyl (trityl), chloroacetyl. The introduction and the removal of these N-protective groups is described herein below. Preferred N-protective group are 2-chloroacetyl and, especially, formyl.

The preferred, non silylated protective groups Pr′ are t-butyl, benzyl, 4-methoxybenzyl, 4-nitrobenzyl, 3,4-dimethoxybenzyl, 2-methoxyethoxymethyl, benzhydryl. In practic, “non silyated” means that Pr′ is other than a —SiAlk₃ group.

The salts of the compounds of formula I in which both R and R′ are hydrogen may be any alkaline metal salt, preferably the sodium or potassium salt, or addition salts with an inorganic or organic acid such as hydrochloric, hydrobromic, sulfuric (sulfate and hydrogenosulfate), methane sulfonic or p-toluene sulfonic acid.

The compounds of formula I above are prepared by a process which is depicted in Scheme 1 below

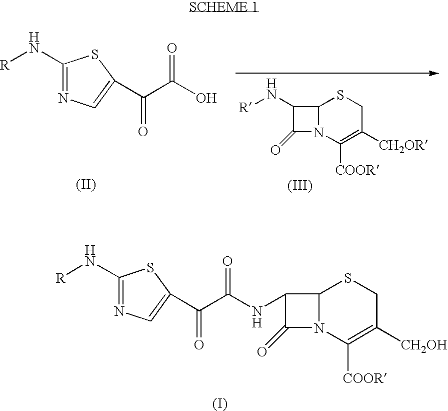

wherein R^{o} is hydrogen or a Alk₃Si group and R″ is R^{o} or a group Pr′, R, R′ Pr′ and Alk being as defined above. In such a process, the starting material of formula II may indifferently be N-protected or unprotected, as it will be explained below.

It is a second object of the present invention to provide a process for the preparation of 7β-(2-aminothiazol-4-yl)glyoxylamido-3-hydroxymethyl-3-cephem-4-carboxylic acid derivatives of formula I above, which comprises reacting an activated 2-aminothiazol-4-ylglyoxylic acid derivative of formula II

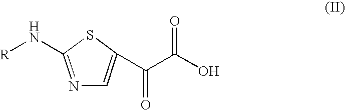

wherein R is hydrogen or a N-protective group Pr, with a 7β-amino-3-hydroxymethyl-3-cephem-4-carboxylic acid derivative of formula III

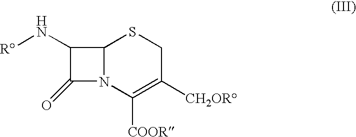

wherein R^{o} is hydrogen or a group Alk₃Si and R″ is R^{o} or a carboxy protective group Pr′ and isolating the of 7β-(2-aminothiazol-4-yl)glyoxylamido-3-hydroxymethyl-3-cephem-4-carboxylic acid derivative thus obtained.

For the use of the compound I obtained at the end of the process as an intermediate in the synthesis of cefotaxime or of ceftriaxone according to the Scheme 1 above, it is suitable that unprotected, N-protected, carboxy-protected and N,carboxy-diprotected 7β-(2-aminothiazol-4-yl)glyoxylamido-3-hydroxymethyl-3-cephem-4-carboxylic acid are available.

If the compound of formula II is used as N-protected derivative, Pr may be any N-protective groups such as t-butoxycarbonyl (Boc), benzyloxycarbonyl (Cbz), benzyloxymethyl, 4-methoxybenzyloxymethyl, benzyl, 4-methoxybenzyl, 3,4-dimethoxybenzyl, formyl, 2-chloroacetyl, 2-bromoacetyl, diphenylmethyl (benzhydryl), triphenylmethyl (trityl) may successfully be used, 2-chloroacetyl and, particularly, formyl, being preferred. If the compound group of formula III is used as carboxy protected derivative, said carboxy group may be silylated (R^{o}=Alk₃Si—, in particular trimethylsilyl) or protected by any protective, non silylated group of the carboxyl function (Pr′) such as, for example, t-butyl, benzyl, benzhydryl, 4-methoxybenzyl, 4-nitrobenzyl, 3,4-dimethoxybenzyl, 2-methoxyethoxymethyl, 4-methoxybenzyl and, particularly, benzhydryl being preferred. Useful N— and carboxy-protective groups are illustrated by T. W. Greene and P. G. M. Wuts in “Protective Groups in Organic Synthesis”, 3^{rd} Edition, 1999, John Wiley & Sons.

The starting compounds of formula II in which R is Pr are mentioned in U.S. Pat. No. 4,201,779 and may be prepared as described in the same document, in particular by reacting a 2,3-dioxobutanoic acid ester with a halogenating agent to obtain a 2,3-dioxo-4-halobutyric acid ester which is treated with thiourea to give the corresponding 2-aminothiazol-4-ylglyoxylic acid ester. This compound may be either treated as described in U.S. Pat. No. 4,201,779 to prepare the N-protected 2-aminothiazol-4-ylglyoxylic acid, by first introducing the Pr group and than saponifying the compound thus obtained, or, according to an embodiment of the present invention, straightforwardly saponified to give the 2-aminothiazol-4-ylglyoxylic acid (formula II, R═R′═H) This free acid may be used as starting material for the process of the present invention, without N-protection, when its benzothiazol-2-ylthioester, the latter described in U.S. Pat. No. 5,484,928, is used as the reactive derivative of the compound of formula II.

According to a preferred embodiment, the benzothiazol-2-ylthioester of the compound of formula II wherein R is hydrogen is prepared according to a new process which is depicted, in a preferred embodiment thereof, in Scheme 2 below

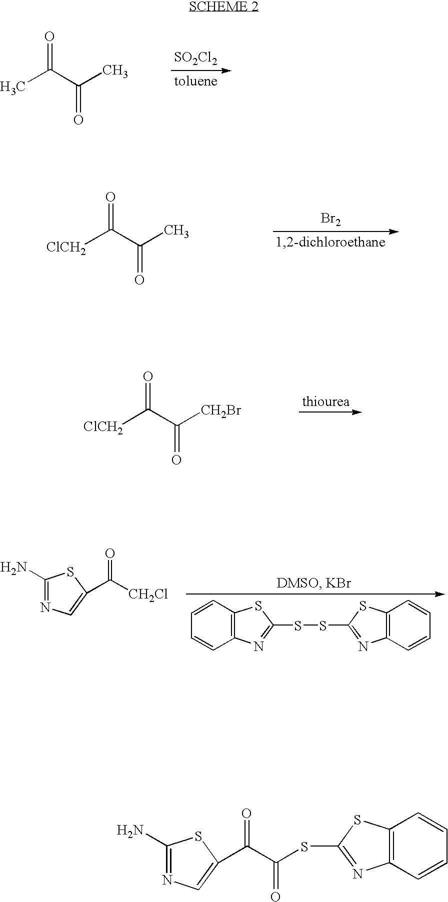

wherein R is as defined above. This new process provides an improved and shorter way, in respect of those described for example in U.S. Pat. No. 4,201,779, U.S. (Toyama) and U.S. Pat. No. 5,484,928, to prepare the benzothiazol-2-ylthioester of (2-aminothiazol-4-yl)glyoxylic acid.

Thus, it is a third object of the present invention to provide a process for the preparation of the benzothiazol-2-ylthioester of (2-aminothiazol-4-yl)glyoxylic acid of formula II′

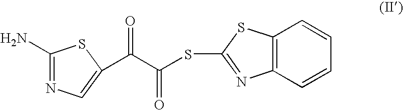

which comprises reacting 1-bromo-3-chlorodibutanone with thiourea and treating the 2-amino 4-(2-chloroacetyl)thiazole thus obtained with bis-(2-benzothiazolyl)disulfide in the presence of dimethyl sufoxide and of an alkaline metal bromide, preferably potassium bromide.

When a compound of formula III wherein R^{o} is Alk₃Si is used as starting material, the silyl derivatives are prepared, generally in situ, starting from the optionally carboxy-protected 7β-amino-3-hydroxymethyl-3-cephem-4-carboxylic acid of formula III′

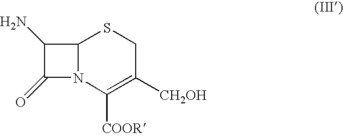

wherein R′ is hydrogen or Pr′ as defined above.

The 7β-amino-3-hydroxymethyl-3-cephem-4-carboxylic acid (III′, R′═H) is a well known compound which is commercially available, easily obtained instead of 7-aminocephalosporanic acid if, during the fermentation in the preparation of cephalosporin C, an esterase is added to the fermentation medium. Its esters are also known.

When, in formula II, R is a Pr group, any functional derivative of the N-protected 2-aminothiazol-4-ylglyoxylic acid may be successfully used. Thus, for example the 2-aminothiazol-4-ylglyoxylic acid ester prepared according to U.S. Pat. No. 4,201,779 is N-protected by reaction with an agent providing the desired protecting groups, for example benzyl chloroformate, N-Cbz-imidazole, dibenzyl carbonate (for introducing Cbz) or their analogs 4-bromobenzyl- and 4-methoxybenzylchloroformate; di-t-butyl dicarbonate or t-butyl chloroformate, t-butylazidoformate, 1-(t-butoxycarbonyl)imidazole, 2-(t-butoxycarbonyloxyimino)-2-phenylacetonitrile, N-(t-butoxycarbonyloxy)phthalimide, N-(t-butylcarbonyloxy)succinimide, 1-(t-butoxycarbonyl)1,2,4-triazole (all for introducing Boc as the Pr group), benzyl bromide, 4-methoxybenzyl chloride or 3,4-dimethoxybenzyl bromide (for introducing an optionally substituted benzyl group as a Pr group); benzyl chloromethyl ether (for introducing a benzyloxymethyl group as the Pr group). These agents for protecting the amino group are illustrated by A. J. Pearson and W. R. Roush in “Handbook of Reagents for Organic Synthesis: Activating Agents and Protecting Groups”, 1999, John Wiley & Sons Ltd. Also 2-chloroacetyl chloride (for introducing the chloroacetyl group, as a Pr group, by reaction with 2-aminothiazol-4-ylglyoxylic acid ester in tetrahydrofuran/dimethylacetamide) and formaldehyde (for introducing the formyl group as the Pr group) are useful N-protecting reagents. The N-protected 2-aminothiazol-4-ylglyoxylic acid ester thus obtained is then saponified to isolate the free acid. The functional derivative of the acid of formula II may be an acid halide thereof, the anhydride, a mixed anhydride, for example with carbonic acid monoethylester or with an alkane or arene sulfonic acid (for example methane sulfonic or p-toluene sulfonic acid, an active ester, an active thioester, for example with mercaptobenzotriazole or the free acid itself, duly activated for example with dicyclohexylcarbodiimide, 1-hydroxybenzotriazole, or with the so-called “BOP reagent”, namely the benzotriazol-1-yloxy-tris(dimethylamino)phosphonium hexafluorophosphate. An advantageous functional derivative is the acid chloride which may be obtained by reaction of the free acid with, for example, thionyl chloride in an inert solvent or using thionyl chloride itself as the solvent. Another advantageous functional derivative is the mixed anhydride with a sulfonic acid, preferably with methane sulfonic acid or p-toluene sulfonic acid, or with a monoester of carbonic acid, preferably with carbonic acid monoethylester, which may be obtained from the N-protected 2-aminothiazol-4-ylglyoxylic acid and an acid chloride such as p-toluenesulfonyl chloride or ethyl chloroformate. The preferred reactive derivative is the benzothiazol-2-yl thioester of formula II′ above.

When, in the formula II, R is hydrogen, it is preferred that the reactive derivative is prepared using reagents which do not affect the free amino group. In this connection 2-aminothiazol-4-ylglyoxylic acid chloride, obtained from the corresponding free acid as described above, and, preferably the above-cited benzothiazol-2-yl thioester of formula II′ are particularly preferred.

The reacting compound of formula III may be the free or carboxy protected 7β-amino-3-hydroxymethyl-3-cephem-4-carboxylic acid (formula III′) which is made to react, for example, with a mixed anhydride of the compound of formula II, in which R is Pr′, with an alkane or arene sulfonic acid such as methane sulfonic acid in an organic solvent in the presence of an organic base.

The reacting compound of formula III may also be a silylated derivative (R^{o}=Alk₃Si, preferably Me₃Si), which is prepared starting from compound III′ and made to react in situ with the reactive derivative of the selected, protected or unprotected, compound of formula II. In general, the compound of formula III′ is suspended in a solvent such as dichlorometane, perchloroethylene, 1,2-dichloroethane, 1,1,1-trichloroethane, tetrayhdrofuran, t-methyl butyl ether, acetonitrile, ethyl acetate or toluene and a silylating agent, such as chlorotrimethylsilane, chlorotriethylsilane, N,O-bis(trimethylsilyl)acetamide, N,N′-bis(trimethylsilyl)urea, cyanotrimethylsilane, hexamethyldisilazane, iodotrimethylsilane, N-(trimethylsilyl)imidazole, trimethylsilyl trifluoromethane sulfonate, is added thereto The mixture is stirred until a solution is formed, then the reactive derivative of compound II is added thereto. The reaction is hexothermic and is controlled by operating at a temperature of from −10 to 25° C. The compound of formula I is isolated according to the conventional techniques, for example by evaporating the solvent and taking up the residue in a solvent in which the compound of formula I precipitates and the compound Alk₃SiOH remains dissolved. In general, alcohols such as methanol, ethanol, isopropanol or 2-methoxyethanol are used as suitable precipitating solvents.

According to a preferred embodiment, the compound of formula III′ is dissolved in an apolar or aprotic polar solvent, in particular in a halogenated, ether, or amide solvent, such as dichloromethane, tetrahydrofurane, dimethylacetamide and treated with hexamethyldisilazane and the mixture thus obtained, consisting of the compound of formula III in which Alk is methyl and R′ is Pr or Me₃Si, is treated with a reactive derivative of the compound of formula II. The optionally N-protected 7β-(2-aminothiazol-4-yl)glyoxylamido-3-hydroxymethyl-3-cephem-4-carboxylic acid derivative of formula I thus isolated is collected by filtration.

When in the compound of formula I thus obtained at least one of the substituents R and R′ is hydrogen, said compound may be isolated in form of a salt thereof or in free form and may optionally converted into an alkaline or acid addition salt thereof. Preferred compounds obtained at the end of the process are either N and carboxy diprotected or unprotected, corresponding to the formulas Ia and Ib

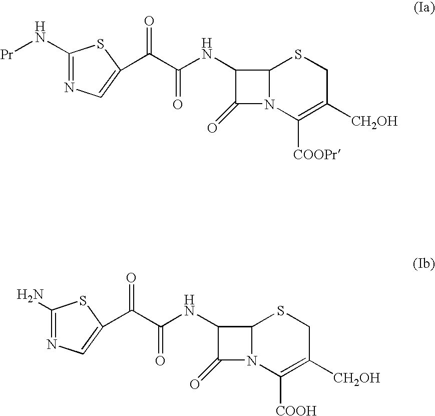

respectively, wherein Pr and Pr′ are as defined above. These compounds are particularly useful intermediates in the synthesis of compounds of formula A above, in particular of cefotaxime and ceftriaxone. The mono-protected derivatives (formula I, R ═H and R′═Pr′ or R═Pr and R′═H) may be obtained by reacting a compound of formula II, in which R═H, with a compound of formula III, in which R″═Pr′, or a compound of formula II, in which R═Pr, with a compound of formula III, in which R″ is SiAlk₃ according to Scheme 1. Alternatively, these mono-protected derivatives may be obtained by submitting the compounds of formulas II and III, in which R is H and R^{o} is Alk₃Si to the process of Scheme 1 and by further protecting either the amino or the carboxy group of the compound of formula Ib thus obtained according to known methods.

The compounds of formula I, in particular the mono- or diprotected ones, preferably those of formula Ia, may be further reacted and converted into the third-generation-cephalosporins of formula A above. Typically, said conversion is carried out by treating said compound of formula Ia with a reactive derivative of a leaving group Y donor; treating the compound thus obtained of formula IVa

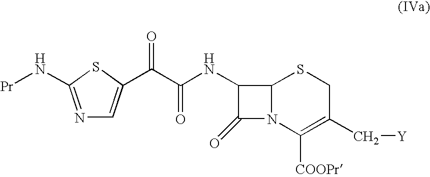

wherein Pr and Pr′ are as defined above and Y is a leaving group, with a nucleophile compound as described in U.S. Pat. No. 5,583,216 in order to replace the leaving group Y with the residue of compound R₄ as defined and illustrated above; treating the compound thus obtained of formula Va

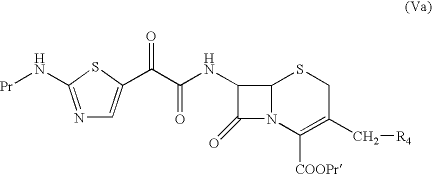

wherein Pr, Pr′ and R₄ are as defined and illustrated above, with an optionally O-protected hydroxylamine of formula R₅—NH₂, wherein R₅ is as defined and illustrated above; and cleaving the Pr and Pr′ protecting groups of the compound thus obtained of formula VIa

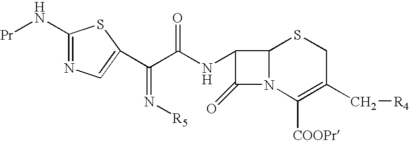

to isolate the compounds of formula A above.

According to a particularly preferred embodiment of the process of the present invention, the compound of formula Ia above is further reacted and converted to cefotaxime or to ceftriaxone by three or four subsequent steps. In particular, the hydroxy group in the 3-position of the cephem moiety is replaced by a leaving group (the term “leaving group” designating an atom, in particular a halogen atom such as chlorine or bromine or a group such as an alkane sulfonyloxy of aryl sulfonyloxy) or into an acetoxy group (which can also act as a leaving group), by substituting the 1,2,5,6-tetrahydro-2-methyl-5,6-dioxo-1,2,4-triazin-3-ylthio group (or, if desired, the acetoxy group by reaction with potassium acetate when the leaving group is an halogen atom such as chlorine) for the leaving group, then reacting the 3-acetoxymethyl- and 3-(1,2,5,6-tetrahydro-2-methyl-5,6dioxo-1,2,4triazin-3-ylthio)methyl derivatives with methoxyamine and finally deprotecting the compounds thus obtained to isolate cefotaxime and ceftriaxone, respectively. A iodine atom as leaving group is useful when a quaternary nucleophilic substituent (R₄), such as a 1-methylpyrrolidinio or a pyridinio must be introduced. The iodomethyl derivatives are prepared by substituting iodine for chlorine or bromine by reaction with sodium iodide.

The three or four steps above occur according to a reaction sequence which, in a preferred embodiment thereof, is depicted in Scheme 3 below,

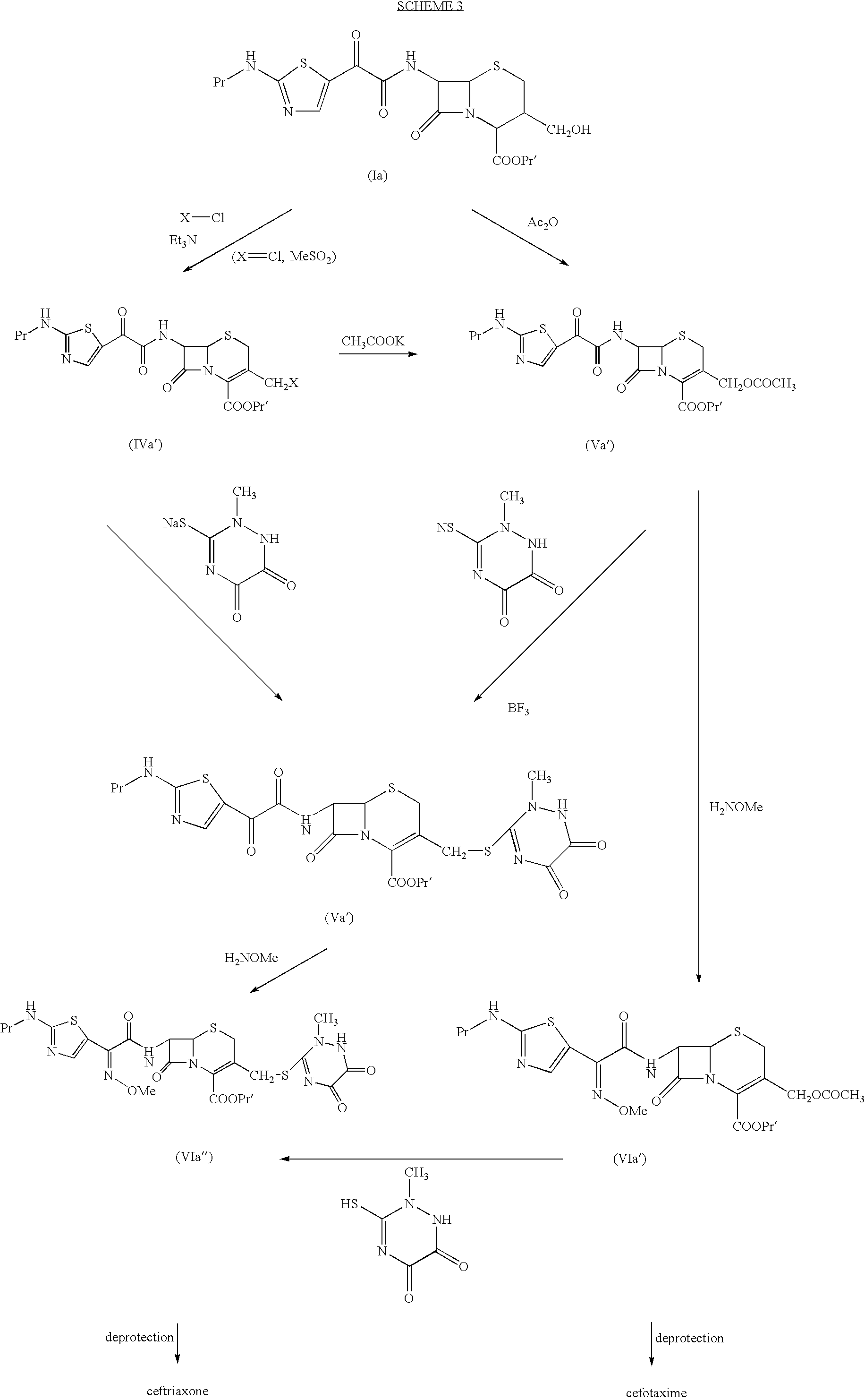

wherein the definition “X—Cl (X═Cl or MeSO₂)” means that, when X is chlorine, the reactant is Cl₂ but the reaction is carried out using a chlorine donor such as, for example, hexachloroacetone and, when X is MeSO₂, the reaction is carried out using the methane sulfonyl chloride to introduce, for example, a methane sulfonyloxy as a leaving group.

It is also understood that Scheme 3 depicts a preferred embodiment and that, for example, the specific reaction sequences shown in Scheme 4 are included in the general reaction sequence

Ia→IVa→Va→VIa→A

illustrated above.

It is a fourth object of the present invention to provide a process as depicted in Scheme 1 above, wherein the 7β-(2-aminothiazol-4-yl)glyoxylamido-3-hydroxymethyl-3-cephem-4-carboxylic acid derivative of formula Ia is further reacted by

(i) treating said compound of formula Ia with a reactive derivative of a leaving group Y donor;

(ii) treating the compound thus obtained of formula IVa

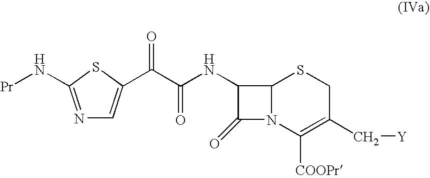

wherein Pr and Pr′ are as defined above and Y is a leaving group, with 1,2,5,6-tetrahydro-2-methyl-5,6-dioxo-1,2,4-triazine-3-thiol (2-methyl-5,6-dioxo-hexahydro-1,2,4triazine-3-thione) or with an alkaline metal salt thereof;

(iii) treating the compound thus obtained of formula Va″

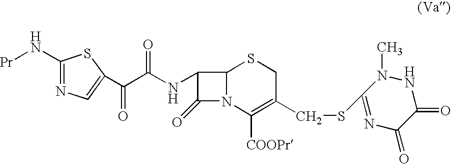

wherein Pr and Pr′ are as defined above, with methoxyamine; and

(iv) cleaving the protecting Pr and Pr′ groups of the compound thus obtained of formula VIa″

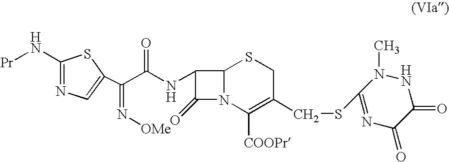

to isolate ceftriaxone.

In step (i) the leaving group donor may be any reactive derivative generating the substituent Y, for example the halide, the anhydride, a mixed anhydride or an active ester of an alkane or arene mono or dicarboxylic acid or of a sulfonic acid, such as acetic anhydride, acetyl chloride, acetyl bromide, propionyl chloride, methane sulfonyl chloride, butane sulfonyl chloride, p-toluene sulfonyl chloride, generating the corresponding acyloxy or sulfonyloxy leaving group, such as the acetoxy, methane sulfonyloxy or p-toluenesulfonyloxy. The leaving group donor may also be an halogen donor, in particular a chlorine or bromine donor, such as a derivative of triphenylphosphine with a chlorine or bromine compound such as triphenylphosphine dichloride (Ph₃PCl₂), triphenylphosphine-tetrachloromethane (Ph₃P—CCl₄), triphenylphosphine-hexachloroacetone, triphenylphosphine-N-chlorosuccinimide, triphenylphosphine dibromide (Ph₃PBr₂), triphenylphosphine-tetrabromomethane (Ph₃P—CCl₄), triphenylphosphine-N-bromosuccinimide. If a iodine atom is needed as leaving group, the iodine is introduced by substituting iodine for chlorine or bromive by reaction with sodium or potassium iodide, according to well known method Bromine, chlorine, acetoxy, methane sulfonyloxy and p-toluene sulfonyloxy groups donors, especially acetic anhydride, methane sulfonyl chloride, p-toluene sulfonyl chloride, triphenylphosphine-N-bromosuccinimide and triphenylphosphine-hexachloroacetone are particularly preferred leaving group donors.

The acetoxy group is introduced under the classic acetylation conditions, by using any acetylating agent, such as, for example, acetic anhydride, acetyl chloride, acetyl bromide, isopropenyl acetate or acetyl cyanide. Typically, 7β-(2-aminothiazol-4-yl)glyoxylamido-3-hydroxymethyl-3-cephem-4-carboxylic acid derivative of formula Ia is treated with acetic anhydride, also used as a solvent. In the case of use of acetyl chloride or acetyl bromide, the acetylation is preferably carried out in a halogenated solvent such as dichloromethane, trichloroethane or 1,1,1-trichloroethane. A catalyst such as pyridine or dimethylaminopyridine may be added to the reaction mixture containing acetic anhydride or the acetyl halide.

The compound of formula IVa thus obtained is isolated according to known techniques.

If an acetoxy group donor, for example acetic anhydride, acetyl chloride or acetyl bromide is used as leaving group donor, the reaction gives a compound of formula Va′

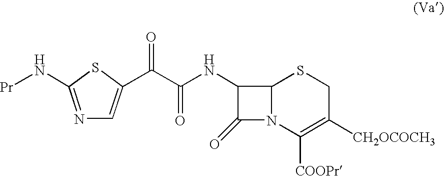

in which Pr and Pr′ are as defined above, which represents a particularly useful intermediate in the preparation of both cefotaxime and ceftriaxone. In fact, compound Va′ has an acetoxy leaving group which can be replaced by the 1,2,5,6-tetrahydro-2-methyl-5,6-dioxo-1,2,4-triazine-3-thio group in the subsequent step (ii), but it can be directly submitted to a treatment with methoxyamine and subsequent deprotection to isolate cefotaxime, as it will be illustrated hereibelow. Compound Va′ may also be obtained by reacting compound IVa, wherein Y is chlorine, with potassium acetate.

In step (ii), the compound of formula IVa is treated with 1,2,5,6-tetrahydro-2-methyl-5,6-dioxo-1,2,4-triazine-3-thiol (2-methyl-5,6-dioxo-hexahydro-1,2,4-triazine-3-thione)

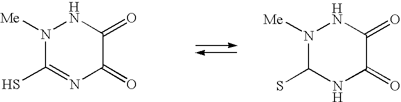

or with an alkaline metal salt thereof in order to replace the leaving group by the 1,2,5,6-tetrahydro-2-methyl-5,6-dioxo-1,2,4-triazine-3-thio group. When the leaving group is the nucleophilic residue of an akane or arene mono or dicarboxylic acid, in particular the acetoxy group (formula Va′), the reaction with 1,2,5,6-tetrahydro-2-methyl-5,6-dioxo-1,2,4-triazine-3-thiol (2-methyl-5,6-dioxo-hexahydro-1,2,4-triazine-3-thione) is carried out in the presence of a Lewis acid, preferably boron trifluoride or boron trifluoride etherate in a polar aprotic solvent such a acetonitrile at a temperature of 20÷25° C. When the leaving group is a halogen atom or an alkane or arene sulfonyloxy group, a compound of formula IVa′

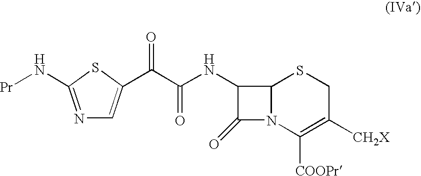

wherein Pr and Pr′ are as defined above and X represent a halogen atom or an alkane or arene sulfonyloxy group, preferably a chlorine atom or a methane sulfonyloxy group is treated with an alkaline metal, preferably sodium, salt of 1,2,5,6-tetrahydro-2-methyl-5,6-dioxo-1,2,4-triazine-3-thiol

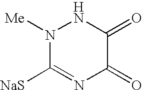

in the presence of a tertiary amine or of a quaternary ammonium salt thereof.

According to a preferred embodiment, a compound of formula IVa′ above, in which X is chlorine, is treated with sodium 1,2,5,6-tetrahydro-2-methyl-5,6-dioxo-1,2,4-triazine-3-thiol in a biphasic water/dichloromethane solvent system in the presence of a tetraalkylammonium halide, for example tetra-n-butylammonium bromide, at a temperature of 20÷25° C. The compound of formula Va″ thus obtained is isolated according to conventional methods, in particular by separating the phases extracting the aqueous one with dichloromethane and recovering the compound Va″ from the collected organic phases.

In step (iii), the compound of formula Va″ is treated with methoxyamine (O-methylhydroxylamine). The reaction is carried out in an alcohol such as methanol or ethanol, in a halogenated solvent, preferably in dichloromethane or in a polar aprotic solvent such as dimethylformamide or dimethylacetamide. Typically, the compound of formula Va″ is treated with methoxyamine in an alcoholic, preferably by methanol, solvent at a temperature of from 20° C. to the refluxing temperature, by further recovery of the obtained product by simple filtration, or in as dimethylformamide or at a temperature of 20÷40° C., by further recovering the obtained product by precipitation with methanol The reaction time is of from 4 to 18 hours.

In step (iv), the compound of formula VIa″ thus obtained is deprotected by cleavage of the Pr and Pr′ groups according to the methods known in the art for the deprotection of primary amines and of carboxylic acids as described for example by T. W. Greene and P. G. M. Wuts in “Protective Groups in Organic Synthesis”, 3^{rd} Edition, 1999, John Wiley & Sons or by A. J. Pearson and W. R. Roush in “Handbook of Reagents for Organic Synthesis: Activating Agents and Protecting Groups”, 1999, John Wiley & Sons Ltd. For example, the N-chloroacetyl group is cleaved by treatment with thiourea as described for example in by M. Masaki et al. in J. Am Chem Soc. 1968, 90, page 4508, by J. E. Baldwin et al. in Tetrahedron, 1986, 42, page 3097 or by T. Allmendinger et al. in Helv. Chim. Acta 1988, 71, 395. According to a preferred embodiment of the process of the present invention, the most advantageous protecting groups are the formyl group for the primary amine on the thiazole moiety and the benzbydryl group for the carboxy radical. These protecting groups are concurrently removed by treatment of the compound of formula VIa″ with anisole in the presence of a Lewis acid or of trifluoroacetic acid, by treatment with an arene sulfonic acid in an arene or by hydrogenolysis catalyzed by Pd/C in an ether solvent such as dioxane, tetrahydrofuran or methyl t-butyl ether.

The ceftriaxone thus obtained is isolated according to the usual procedures and purified for its use as active ingredient for pharmaceutical compositions.

When, at the end of step (i) a compound of formula Va′ above is obtained, it is converted into cefotaxime by submitting said compound Va′ to two steps only.

Thus, it is a fifth object of the present invention to provide a process as depicted in Scheme 1 above, wherein the 7β-(2-aminothiazol-4-yl)glyoxylamido-3-hydroxymethyl-3-cephem-4-carboxylic acid derivative of formula Ia is further reacted by

(a) treating said 7β-(2-arninothazol-4-yl)glyoxylamido-3-hydroxymethyl-3-cephem-4-carboxylic acid derivative of formula Ia with an acetylating agent;

(b) treating the 7β-(2-aminothiazol-4-yl)glyoxylamidocephalosporanic acid derivative thus obtained of formula Va′ with methoxyamine, and

(c) cleaving the protecting Pr and Pr′ groups of the compound thus obtained of formula VIa′

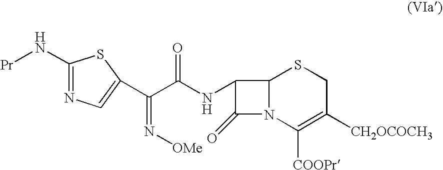

wherein Pr and Pr′ are as defined above, to isolate cefotaxime.

Step (a) is carried out under the acetylation conditions illustrated for step (i) above and the compound of formula V′a is isolated accordingly or directly submitted to step (b), especially when acetic anhydride is used as both reacting agent and solvent.

Step (b) is also carried out as illustrated for step (iii) above, by reacting the compound of formula Va′ thus obtained with methoxyamine in an alcoholic, preferably by methanol, solvent or in a polar aprotic solvent. If the compound of formula V′a is prepared by using acetic anhydride both as a solvent and as reactant, it is possible to carry out the reaction in glacial acetic acid and water. At the end of the reaction, which is carried out at a temperature of from −10° C. to room temperature, the compound of formula VI′a thus obtained is isolated according to conventional procedures, for example by precipitation with water or with cold methanol.

Step (c), consisting of the cleavage of the Pr and Pr′ groups, is carried out as described for the removal of the protecting groups Pr and Pr′ of compound Va″ in the above step (iv). The cefotaxime thus obtained is isolated according to the usual procedures and purified for its use as active ingredient for pharmaceutical compositions.

The compound of formula VIa′ obtained at the end of step (b) above may also be submitted to a reaction with 1,2,5,6-tetrahydro-2-methyl-5,6-dioxo-1,2,4-triazine-3-thiol (2-methyl-5,6-dioxo-hexahydro-1,2,4-triazine-3-thione) in the presence of a Lewis acid, preferably of boron trifluoride or boron trifluoride etherate in a polar aprotic solvent such a acetonitrile at a temperature of 20÷25° C., i.e. under the conditions of step (ii) above for the conversion of compound Va′ into compound Va″.

Thus, it is a sixth object of the present invention to provide a process as depicted in Scheme 1, wherein the 7β-(2-aminothiazol-4-yl)glyoxylamido-3-hydroxymethyl-3-cephem-4-carboxylic acid derivative of formula Ia is further reacted by

(i′) treating said 7β-(2-aminothiazol-4-yl)glyoxylamido-3-hydroxymethyl-3-cephem-4-carboxylic acid derivative of formula Ia with an acetylating agent; then

(ii′-iii′) submitting the compound of formula Va′ thus obtained

to a reaction with 1,2,5,6-tetrahydro-2-methyl-5,6-dioxo-1,2,4-triazine-3-thiol (2-methyl-5,6-dioxo-hexahydro-1,2,4triazine-3-thione) in the presence of a Lewis acid, and

to a reaction with methoxyamine in any order; and, finally,

(iv′) cleaving the protecting Pr and Pr′ groups of the compound of formula VIa″ thus obtained to isolate ceftriaxone.

Step (i′) is identical with step (i) above. Steps (ii′) and (iii′) may be carried out in any order, i.e. either by first treating compound of formula Va′ with 1,2,5,6-tetrahydro-2-methyl-5,6-dioxo-1,2,4-triazine-3-thiol (2-methyl-5,6dioxo-hexahydro-1,2,4-triazine-3-thione) in the presence of a Lewis acid under the conditions illustrated above for step (ii) and, then, by reacting the compound of formula Va″ thus obtained with methoxyamine under the conditions of step (iii) above or by first treating compound of formula Va′ with methoxyamine under the conditions of step (b) above and then reacting the compound of formula VI′a thus obtained with 1,2,5,6-tetrahydro-2-methyl-5,6-dioxo-1,2,4-triazine-3-thiol (2-methyl-5,6dioxo-hexahydro-1,2,4-triazine-3-thione) in the presence of a Lewis acid under the conditions illustrated above for step (ii).

Thus, the present invention provide a versatile process for preparing third-generation-cephalosporins of formula A above, in particular cefotaxime and ceftriaxone in few steps using the easily obtainable intermediate of formula I above.

The following examples illustrate the invention without, however, limiting it.

### EXAMPLE 1

To 270 ml of ethyl acetate, 0.102 mol of (2-tritylaminothiazol-4-yl)glyoxylic acid (U.S. Pat. No. 4,201,779) are added under stirring at a temperature of −10° C., then 0.109 mol of methane sulfonyl chloride are added into the mixture thus obtained. The temperature of the mixture is brought to −15° C. and 0.121 mol of diisopropylamine are slowly added thereinto, by keeping the temperature not higher than about 0° C. The mixture is stirred at a temperature of from −5° C. to 0° C. for 30 minutes, then is cooled to −10° C. The solution thus obtained (Solution A), containing the (2-tritylaminothiazol-4-yl)glyoxyloyl methane sulfonyl mixed anhydride is added, in 15 minutes and at −10° C. to a Solution B (previously prepared by adding 0.078 mol of 7β-amino-3-hydroxymethyl-3-cephem-4-carboxylic acid to 54 ml methanol at −10° C., treating the solution thus obtained with 0.172 mol of diisopropylamine and stirring the solution 30 minutes at about 5° C.). At the end of the reaction, the suspension is concentrated under reduced pressure at about 15° C., then it is treated with 90 ml of ethyl acetate and, then, the operation of concentration and taking up with the ethyl acetate is repeated four times again to obtain a suspension containing the diisopropylamine salt of 7β-(2-tritylaminothiazol-4-yl)glyoxylamido-3-hydroxymethyl-3-cephem-4-carboxylic acid, which is separated by filtration.

1. A 7β-(2-aminothiazol-4-yl)glyoxylamido-3-hydroxymethyl-3-cephem-4-carboxylic acid derivative of formula I 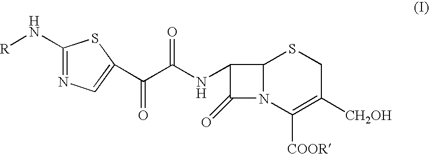
wherein R is hydrogen or a N-protective group Pr and R′ is hydrogen or a non-silylated carboxy protective group Pr′, or a salt of said derivatives of formula I in which at least one of R and R′ is hydrogen.

2. The 7β-(2-aminothiazol-4-yl)glyoxylamido-3-hydroxymethyl-3-cephem-4-carboxylic acid derivative of claim 1 having the formula I wherein both of R and R′ are hydrogen, or a salt thereof with an alkaline metal or with an organic base.

3. The 7β-(2-aminothiazol-4-yl)glyoxylamido-3-hydroxymetyl-3-cephem-4-carboxylic acid derivative of claim 1 having the formula I, wherein R is a N-protective group and R′ is a carboxy protective group.

4. The 7β-(2-aminothiazol-4-yl)glyoxylamido-3-hydroxymethyl-3-cephem-4-carboxylic acid derivative of claim 3 wherein said N-protective group is formyl or chloroacetyl and said carboxy protective group is p-methoxybenzyl or benzhydryl.

5. The 7β-(2-aminothiazol-4-yl)glyoxylamido-3-hydroxymethyl-3-cephem-4-carboxylic acid derivative of claim 4, wherein said N-protective group is formyl and said carboxy protective group is benzhydryl.

6. A process for the preparation of 7β-(2-aminothiazol-4-yl)glyoxylamido-3-hydroxymethyl-3-cephem-4-carboxylic acid derivatives of formula I 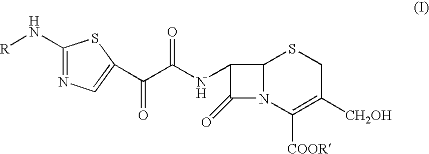
which comprises reacting an activated 2-aminothiazol-4-ylglyoxylic acid derivative of formula II 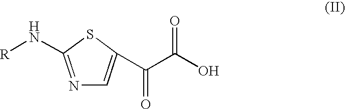
wherein R is hydrogen or a N-protective group, with a 7β-amino-3-hydroxymethyl-3-cephem-4-carboxylic acid derivative of formula III 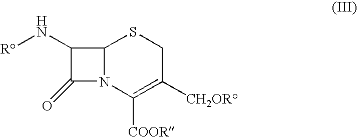
wherein R^{o} is hydrogen or a group Alk₃Si and R″ is R^{o} or a carboxy protective group Pr′ and isolating the of 7β-(2-aminothiazol-4-yl)glyoxylamido-3-hydroxymethyl-3-cephem-4-carboxylic acid derivative thus obtained.

7. The process of claim 6, wherein said 2-aminothiazol-4-ylglyoxylic acid derivative to be activated has the formula II in which R is hydrogen and said 7β-amino-3-hydroxymethyl-3-cephem-4-carboxylic acid derivative has the formula III in which each of R^{o} and R″ is a trimethylsilyl group, whereby a compound of formula Ib 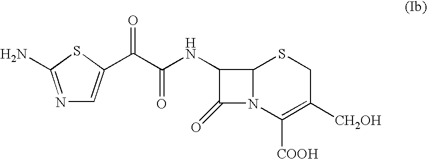
is isolated.

8. The process of claim 7, wherein said 2-aminothiazol-4-ylglyoxylic acid derivative is the benzothiazol-2-ylthioester of (2-aminothiazol-4-yl)glyoxylic acid of formula II′

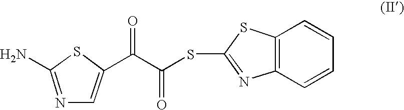

9. The process of claim 8, wherein said 2-aminothiazol-4-ylglyoxylic acid derivative is the benzothiazol-2-ylthioester of (2-aminothiazol4-yl)glyoxylic acid of formula II′ is previously prepared by a process which comprises reacting 1-bromo-3-chlorodibutanone with thiourea and treating the 2-amino 4-(2-chloroacetyl)thiazole thus obtained with bis-(2-benzothiazolyl)disulfide in the presence of dimethyl sulfoxide and of an alkaline metal bromide.

10. The process of claim 9, wherein said alkaline metal bromide is potassium bromide.

11. The process of claim 6, wherein said 2-aminothiazol-4-ylglyoxylic acid derivative to be activated has the formula II in which R is a N-protecting group Pr and said 7β-amino-3-hydroxymethyl-3-cephem-4carboxylic acid derivative has the formula III in which R^{o} is trimethylsilyl and R″ is a non-silylated carboxy protecting group Pr′, whereby a compound of formula Ia 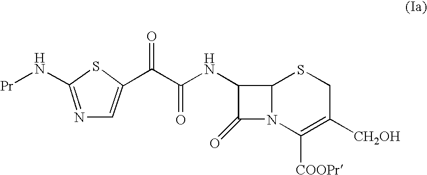
wherein Pr and Pr′ are as defined above, is isolated.

12. The process of claim 11, wherein in formula II Pr is formyl or 2-chloroacetyl and, in formula III, R″ is benzhydryl or p-methoxybenzyl.

13. The process of claim 12, wherein Pr is formyl and in formula III R″ is benzhydryl.

14. The process of claim 11, wherein said 7β-(2-aminothiazol-4-yl)glyoxylamido-3-hydroxymethyl-3-cephem-4-carboxylic acid derivative of formula Ia is further reacted by
(i) treating said compound of formula Ia with a reactive derivative of a leaving group Y donor;
(ii) treating the compound thus obtained of formula IVa 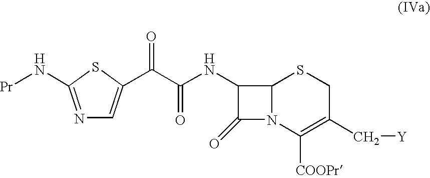
wherein Pr and Pr′ are as defined above and Y is a leaving group, with 1,2,5,6-tetrahydro-2-methyl-5,6-dioxo-1,2,4-triazine-3-thiol (2-methyl-5,6-dioxo-hexahydro-1,2,4-triazine-3-thione) or with an alkaline metal salt thereof;
(iii) treating the compound thus obtained of formula Va″ 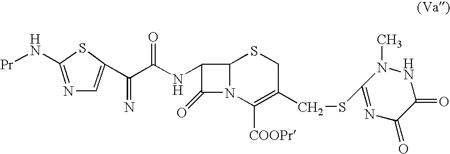
wherein Pr and Pr′ are as defined above, with methoxyamine; and
(iv) cleaving the protecting Pr and Pr′ groups of the compound thus obtained of formula VIa″ 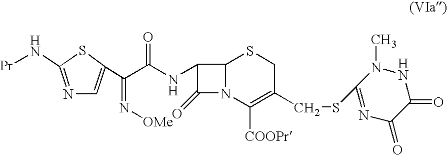
to isolate ceftriaxone.

15. The process of claim 11, wherein said 7β-(2-aminothiazol-4-yl)glyoxylamido-3-hydroxymethyl-3-cephem-4-carboxylic acid derivative of formula Ia is further reacted by
(a) treating said 7β-(2-aminothiazol-4-yl)glyoxylamido-3-hydroxymethyl-3-cephem-4-carboxylic acid derivative of formula Ia with an acetylating agent;
(b) treating the 7β-(2-aminothiazol-4-yl)glyoxylamidocephalosporanic acid derivative thus obtained of formula Va′ 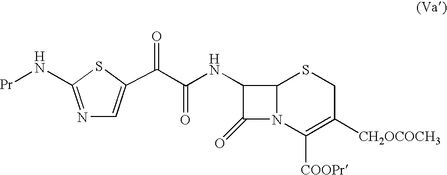
wherein Pr and Pr′ are as defined in claim 11, with methoxyamine; and
(c) cleaving the protecting Pr and Pr′ groups of the compound thus obtained of formula VIa′ 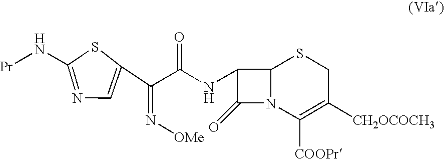
wherein Pr and Pr′ are as defined above, to isolate cefotaxime.

16. The process of claim 11, wherein said 7β-(2-aminothiazol-4-yl)glyoxylamido-3-hydroxymethyl-3-cephem-4-carboxylic acid derivative of formula Ia is further reacted by
(i′) treating said 7β-(2-aminothiazol-4-yl)glyoxylamido-3-hydoxymethyl-3-cephem-4-carboxylic acid derivative of formula Ia with an acetylating agent; then
(ii′-iii′) submitting the compound thus obtained of formula Va′ 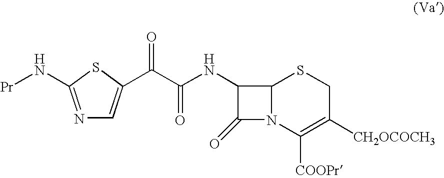
to a reaction with 1,2,5,6-tetrahydro-2-methyl-5,6-dioxo-1,2,4-triazine-3-thiol (2-methyl-5,6-dioxo-hexahydro-1,2,4-triazine-3-thione) in the presence of a Lewis acid, and
to a reaction with methoxyamine in any order; and, finally,
(iv′) cleaving the protecting Pr and Pr′ groups of the compound thus obtained of formula VIa″ 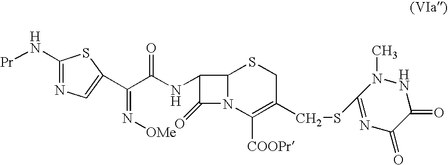
to isolate ceftriaxone.
